(19) United States
(12) Patent Application Publication
(10) Pub. No.: US 2012/0148512 A1
(43) Pub. Date: Jun. 14, 2012

(54) Method for Increasing the Sun Protection Factor of a Cosmetic and/or Dermatological Preparation

(75) Inventor: Heike Flösser-Müller, Ludwigshafen (DE)

(73) Assignee: BASF SE, Ludwigshafen (DE)

(21) Appl. No.: 13/402,522

(22) Filed: Feb. 22, 2012

Related U.S. Application Data
(63) Continuation of application No. 12/306,098, filed on Dec. 22, 2008, now abandoned.

(30) Foreign Application Priority Data
Jun. 23, 2006 (EP) 06115950.5

(51) Int. Cl. A61K 8/49; A61Q 17/04

(52) U.S. Cl. 424/59

(57) ABSTRACT

The present invention relates to a method of increasing the sun protection factor of a cosmetic and/or dermatological preparation, to the use of UV-A filters for increasing the sun protection factor of such a preparation and to specific photoprotective cosmetic and/or dermatological preparations.

The present invention relates to a method of increasing the sun protection factor of a cosmetic and/or dermatological preparation, to the use of UV-A filters for increasing the sun protection factor of such a preparation and to specific photoprotective cosmetic and/or dermatological preparations.

The photoprotective agents used in cosmetic and/or dermatological preparations have the task of reducing the harmful effects of sunlight, i.e. in particular those of UV radiation in sunlight, on the human skin (Chemie in unserer Zeit, 2004, 38, 98-112). In addition, however, these photoprotective agents also serve to protect the ingredients of the formulation against decomposition or degradation by UV radiation. The use of photoprotective filter substances in cosmetic compositions for protecting the human skin is regulated by law in most industrialized countries through positive lists with maximum values for the use of such substances.

UV radiation is divided, according to wavelength, into UV-A light (320-400 nm) and UV-B light (280-320 nm) and UV-C light (100-280 nm).

Rays with a wavelength of less than 290 nm are absorbed by the ozone layer in the earth's atmosphere and do not reach the earth. UV rays in the range between 290 nm and 320 nm, belonging to the so-called UV-B region, can cause, for example, an erythema on the skin, i.e. sunburn, or even burns of greater or lesser severity. The relatively narrow region around 308 nm is stated as a maximum of the erythema effectiveness of sunlight.

The sun protection factor (SPF) indicates how much longer the skin protected with the photoprotective agent can be exposed to solar radiation before the same erythema reaction arises as in the case of unprotected skin (i.e. ten times as long compared to unprotected skin for SPF=10). The sun protection factor is thus in particular a measure of the protection against UV-B radiation. It is compulsory for the marketing of sunscreen products to state the SPF in vivo, i.e. measured on people. In Europe, South America and Japan, the protocol of the “International SPF Test Method 2003” regulates the determination of the SPF in vivo.

Numerous compounds are known for protecting against UV-B radiation; these are, inter alia, triazine derivatives, derivatives of 3-benzylidenecamphor, of 4-aminobenzoic acid, of cinnamic acid, of salicylic acid, of benzophenone, and of 2-phenylbenzimidazole.

UV-A radiation, the range between 320 nm and 400 nm, also has a product-damaging as well as a skin-damaging effect, and it is thus likewise important to have available UV-A filter substances in cosmetic and dermatological formulations. It has for a long time been incorrectly assumed that the long-wave UV-A radiation has only a negligible biological effect. In contrast to UV-B light, which only penetrates as far as the epidermis (outer skin), UV-A radiation passes into the deep layers of the dermis (corium), where, for example, blood vessels are also located. In addition, UV-A light, likewise in contrast to UV-B light, is able to penetrate glass windows and e.g. car windscreens, meaning that a person is not protected even inside buildings or in the car.

In the meantime, numerous studies have shown that UV-A radiation is at least equally as dangerous as UV-B radiation with regard to the triggering of photodynamic, specifically photoallergic and phototoxic, reactions and chronic changes in the skin. Thus, it has, inter alia, been found that even small but regular doses of UV-A radiation, as occur under quite normal everyday conditions, are enough to significantly damage, for example, the connective tissue of the skin, more accurately the collagen and elastin fibers. This skin damage, together with other chronic light-induced changes in the skin, is generally known under the term “premature skin aging” or “photoaging”. The clinical symptoms of skin aged prematurely by light include, for example, lines and wrinkles, reduced elasticity and strength, and reduced moisture. In addition, the areas affected by photoinduced skin aging can have irregular pigmentation (e.g. “age spots”) and keratoses.

UV-A radiation is also able to damage the DNA, which in the worst case scenario can lead to skin cancer.

Approximately 90% of the ultraviolet radiation which reaches the earth consists of UV-A rays. Whereas UV-B radiation varies considerably depending on a large number of factors (for example season and time of day or latitude), UV-A radiation remains relatively constant from day to day irrespective of seasonal and diurnal or geographic factors. It is therefore particularly important to use UV-A filters not only in pure sunscreen preparations, but also in day care, such as also in decorative cosmetics, in order to protect the skin against chronic skin damage, as results from slight but regular UV-A exposure, and to counteract, for example, premature skin aging.

In general, the photoabsorption behavior of photoprotective filter substances is very well known and documented. For the dosing of the substances in the finished formulations, the absorption spectra on their own can at best be a guide. Interactions of the UV filter substances with one another, with ingredients of the formulation or with the skin itself can have unforeseeable effects on the protective effect. In addition, it is generally difficult to estimate beforehand how evenly and in what layer thickness the filter substances are spread in and on the horny layer of the skin. All this thus has effects on the photoprotective effect of the preparations and makes an exact forecast difficult or impossible.

For testing UV-A protection, for most countries there is hitherto still no harmonized and generally binding method. However, the PPD method is usually used (PPD=persistent pigment darkening). This is the legal basis in Japan (Japan Cosmetic Industry Association (JCIA); Measurement standard for UVA protection efficacy. Jan. 1, 1996.). Here—similarly to the determination of the sun protection factor—a value is ascertained which indicates how much longer the skin protected with the photoprotective agent can be irradiated with UV-A radiation before the same pigmentation arises as in the case of unprotected skin.

Another established classification of the UV-A protection of a sunscreen formulation used throughout Europe is the so-called Australian Standard (AS/NZS 2604:1997). Here, the transmission of the preparation in the UV-A region is measured. In order to satisfy the standard, the preparation must transmit at most 10% of the incident UV-A radiation in the range 320 to 360 nm, i.e. it must block at least 90% of the radiation in this range.

In the past, the main issue in sun protection was the avoidance of sunburn, i.e. protection against erythemogenic UV-B radiation, expressed in ever higher SPF values, even up to SPF>100. In products from the past, no or only inadequate UV-A protection was often present in these products.

With increasing knowledge about the harmful effect of UV-A radiation, the need for increased use of UV-A filters for skin protection also becomes obvious and regulation for a minimum UV-A protection in sunscreen products becomes necessary. This is reflected by the currently increased activities on the theme of minimum amount of UV-A protection and laying down a harmonized UV-A determination method in sunscreen compositions on the part of industry (COLIPA) and also the EU Commission.

To protect against UV-A rays, use is often made of derivatives of dibenzoylmethane, although their photostability is inadequate (Int. J. Cosm. Science 10, 53, 1988).

EP-A-1 046 391 describes the use of amino substituted hydroxybenzophenones as photostable UV-A filters in cosmetic preparations.

Since the erythemogenicity of UV-B light is up to 1000 times greater than that of UV-A light, and the sun protection factor (SPF) indicates a measure of the protection against erythema, the amount of substances which have their absorption maximum in the UV-B region (UV-B filters) of a sunscreen formulation is usually increased to increase the protection against sunburn and thus to increase the sun protection factor.

By contrast, UV-A filters are predominantly used to protect against the abovementioned consequences of UV-A light.

The term UV protection is understood as meaning both the protection against UV-A radiation and also against UV-B radiation.

WO 2005/094772 describes, for example, a day cream which comprises, as the sole UV filter, hexyl 2-(4-diethylamino-2-hydroxybenzoyl)benzoate, which is a photostable UV-A filter. The described day cream reduces tanning of the skin through solar irradiation. Sun protection factors are not stated.

EP-A-1 290 999 describes cosmetic sticks with very good properties with regard to stickiness, skin compatibility and skin care benefits which, besides the UV-A filter hexyl 2-(4-diethylamino-2-hydroxybenzoyl)benzoate, comprise further UV filter substances. Example 4 describes a stick formulation which comprises a higher fraction of UV-A filters than the sum of UV-B and/or broadband filters, while in each of the other formulations the sum of the weight fractions of UV-A filters is lower than the sum of the weight fractions of UV-B and/or broadband filters. Sun protection factors are not stated.

In the Proceedings of the 21^{st} IFSCC International Congress 2000, Berlin, page 530 ff, a positive effect is described which the addition of small amounts of a UV-A filter (BMDBM) brings about on the SPF. The mass ratio of the UV-A filters to that of the UV-B filters used here was considerably less than 0.5.

WO 03/039507 describes photoprotective formulations which comprise, as UV-A filter, in particular hexyl 2-(4-diethylamino-2-hydroxybenzoyl)benzoate and, as UV-B and/or broadband filter, at least one sparingly soluble triazine derivative and/or benzotriazine derivative. No values for sun protection factors are stated for the various formulations.

There continues to be a need for ever more effective sunscreen formulations which reduce both the effects of sunburn-causing UV-B radiation and also the effects of UV-A radiation which, inter alia, increases the rate of premature skin aging (“photoaging”). Here, as efficient as possible a utilization of the available filter substances should be achieved, i.e. a high sun protection effect with the smallest possible amount of photoprotective filter substance in order to avoid unnecessary burdening in particular to the skin, but also to the environment with chemicals. Furthermore, a lower mass fraction of efficient UV filters increases the flexibility of the formulator to use, for example, larger or other fractions of cosmetic ingredients. It is further desirable to achieve the desired effect using a small number of photoprotective filter substances in a formulation. This reduces the complexity of a cosmetic or dermatological formulation. It is thus the aim to achieve the highest possible UV-A protection and also high UV-B protection using as little UV filter substance as possible.

It was an object of the present invention to indicate possible ways as to how, in particular, the erythema protection (expressed as SPF) can be increased with efficient utilization of the photoprotective filter substances used and at the same time how very good UV-A protection is achieved.

This object is achieved by a method of increasing the sun protection factor of a cosmetic and/or dermatological preparation which comprises at least one UV-B and/or broadband filter by adding at least one UV-A filter during the production of the preparation, wherein, in the finished cosmetic and/or dermatological preparation, a value for the ratio of the sum of the mass of the UV-A filters to the sum of the mass of the UV-B filters and broadband filters of at least 0.5 is established.

The sun protection factor (SPF) is, as explained above, a measure of the protection against UV-B radiation.

According to the invention, increasing the sun protection factor of a cosmetic and/or dermatological preparation means achieving a higher SPF determined by the International SPF method (2003) known to the person skilled in the art by at least 1 to 3, preferably by at least 4 to 6, in particular by at least 7 to 9 SPF units, depending on the starting value and amount of added UV-A filter, through the method according to the invention.

Preferably, in the method according to the invention, an increase by at least 1, preferably at least 1.3 SPF units while keeping the sum of the weight fractions of the UV-B filters and/or broadband filters in the formulation constant is achieved by increasing the UV-A filter by 1% by weight based on the mass of the finished preparation.

In general, cosmetic and/or dermatological preparations are understood as meaning mixtures or formulations which are used for topical application to skin or hair and which are suitable (i) for preventing damage to human skin and/or human hair, (ii) for treating existing damage to human skin and/or human hair, (iii) for caring for human skin and/or human hair and/or (iv) for improving the feel of the skin (sensory properties). Compositions for decorative cosmetics are explicitly included. The cosmetic and/or dermatological preparations described in the method according to the invention are preparations whose main indication is primarily (e.g. in the case of sunscreen preparations) but also inter alia (e.g. in the case of day care products, in the case of antiaging products, in the case of self-tanning preparations) protection of the skin against damage by sunlight, specifically by UV-B radiation (280 to 320 nm) and UV-A radiation (>320 nm). Besides the UV filter substances, the cosmetic and/or dermatological preparations comprise, in a cosmetically compatible medium, suitable auxiliaries and additives which are chosen with regard to the specific field of use. Auxiliaries and additives of this type are known to the person skilled in the art and can be found, for example, in handbooks of cosmetics, for example Schrader, Grundlagen and Rezepturen der Kosmetika [Fundamentals and formulations of cosmetics], Hüthig Verlag, Heidelberg, 1989, ISBN 3-7785-1491-1, or Umbach, Kosmetik: Entwicklung, Herstellung und Anwendung kosmetischer Mittel [Cosmetics: development, preparation and use of cosmetic compositions], 2nd expanded edition, 1995, Georg Thieme Verlag, ISBN 3 13 712602 9.

The term UV-A filters is understood as meaning in particular oil-soluble or water-soluble substances, but also sparingly soluble or pigment-like substances whose absorption maximum is in the UV-A region, i.e. in the range between 320 and 400 nm. As UV-A filters, preference is given to using substances whose absorption maximum is between 330 and 400 nm, in particular between 350 and 380 nm.

In addition, particular preference is given to those UV-A filters which are themselves photostable or are photostabilized by further substances.

In addition, in the method according to the invention, as UV-A filters, preference is given to using those substances which have a high specific absorption in the UV-A region, preferably a specific absorption of A 1%/1 cm (1% strength solution at 1 cm path length) at least 700, in particular of at least 900.

Examples of highly absorbent UV-A filters are dibenzoylmethane derivatives, in particular 4-(tert-butyl)-4′-methoxydibenzoylmethane (BMDBM, CAS No. 70356-09-1) with an absorption maximum at 356 nm, which is sold by DSM under the brand Parsol 1789 and by Merck under the trade name Eusolex® 9020, and is characterized by the following structure:

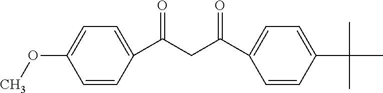

A further dibenzoylmethane derivative is 4-isopropyldibenzoylmethane (CAS No. 63250-25-9), which is sold by Merck under the name Eusolex® 8020. Eusolex® 8020 is characterized by the following structure:

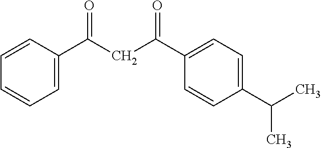

The dibenzoylmethane derivative 4-(tert-butyl)-4′-methoxydibenzoylmethane is itself not photostable and is therefore advantageously used for the purpose of stabilization together with other UV filters which act as triplet quenchers, such as, for example, octocrylene or 3-(4′-methylbenzylidene)-DL-camphor, or with pure triplet quenchers such as diethylhexyl naphthalate (Hallbrite TQ from CP Hall or Corapan TQ from Symrise) or diethylhexyl syringylidene malonate (Oxynex ST from Merck) or in the presence of manganese-doped TiO₂ (for example Optisol™ from Oxonica).

Further examples of advantageous UV-A filters are amino substituted hydroxybenzophenones with the following formula

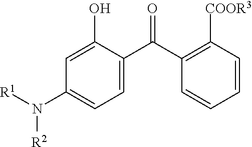

in which

R¹ and R², independently of one another, are hydrogen, C₁-C₂₀-alkyl, C₃-C₁₀-cycloalkyl or C₃-C₁₀-cycloalkenyl, where the substituents R¹ and R², together with the nitrogen atom to which they are bonded, can form a 5- or 6-membered ring and

R³ is a C₁-C₂₀-alkyl radical.

A particularly advantageous highly absorbent amino substituted hydroxybenzophenone for the purposes of the present invention is hexyl 2-(4-diethylamino-2-hydroxybenzoyl)benzoate (DHHB) with an absorption maximum at 354 nm, which has the following structure:

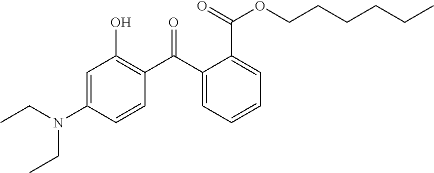

This compound is sold under the name Uvinul® A Plus by BASF AG. One example of water-soluble UV-A filters are sulfonated compounds such as phenylene-1,4-bis(2-benzimidazyl)-3,3′-5,5′-tetrasulfonic acid, which is characterized by the following structure:

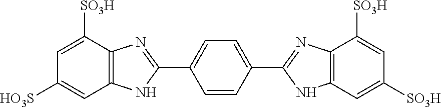

and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Disodium Phenyldibenzimidazole Tetrasulfonate (DPDT) (CAS No.: 180898-37-7), which is available under the trade name Neo Heliopan® AP from Symrise.

A further sulfonated UV-A filter is 3,3′-(1,4-phenylenedimethine)bis(7,7-dimethylylmethanesulfonic acid 2-oxo-bicyclo[2.2.1] heptane-1, and its sodium, potassium or triethanolammonium salt

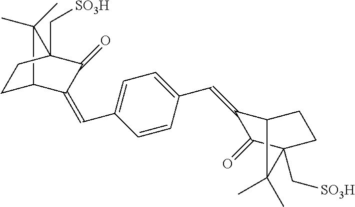

with the INCI name Terephthalidene Dicamphor Sulfonic Acid (TDSA, CAS. No.: 90457-82-2), which is produced, for example, under the trade name Mexoryl® SX by Chimex.

The UV-A filters preferably used in the method according to the invention are photostable and/or photostabilized UV-A filters—alone or in combinations with one another, in particular those with a high specific absorption of at least 700, in particular of at least 900.

A photostable UV-A filter is understood as meaning a filter substance which, under realistic irradiation conditions, does not lose, or loses only slightly, its ability to absorb (e.g. up to 10% loss after sun-simulated irradiation over 10 standard MED). MED is understood as meaning the minimum erythemogenic radiation dose, i.e. the radiation dose which causes the first signs of an erythema, i.e. a slight reddening, on unprotected skin. The MED varies from individual to individual. An erythemogenic radiation dose of 250 J/m² is defined as standard MED.

One method of determining photostability is described by Berset et al. in the International Journal of Cosmetic Science 1996, page 167-177. Retaining more than 90% of the absorption in the absorption maximum following sun-simulated irradiation (5 and 10 MED), 2-phenylbenzimidazole-5-sulfonic acid, for example, is classified as photostable.

A photostabilized UV-A filter is a filter which on its own largely or completely loses its ability to absorb upon exposure, but in combination with suitable further substances largely retains its ability to absorb upon exposure.

Examples of photostable UV-A filters are hexyl 2-(4-diethylamino-2-hydroxybenzoyl)benzoate (Uvinul® A Plus), Terephthalidene Dicamphor Sulfonic Acid (TDSA) or Disodium Phenyldibenzimidazole Tetrasulfonate (DPDT) and an example of a photostabilized UV-A filter is BMDBM stabilized, for example, with octocrylene or diethylhexylnaphthalate (e.g. Hallbrite TQ).

The UV-A-filter used is particularly preferably an amino substituted hydroxybenzophenone, as described, for example, in EP 1 046 391 A2. As highly absorbent UV-A filter, very particular preference is given to using hexyl 2-(4-diethylamino-2-hydroxybenzoyl)benzoate, especially as the sole UV-A filter.

The term UV-B filters is understood as meaning oil-soluble, water-soluble, sparingly soluble or pigment-like substances whose absorption maximum is in the UV-B region, i.e. in the range between 290 and 320 nm.

The term broadband filters is understood as meaning organic or inorganic oil-soluble, water-soluble, sparingly soluble or pigment-like substances which have marked absorption both in the UV-A and in the UV-B region, or a largely constant absorption which transfers smoothly from the UV-B region to the UV-A region.

Examples of oil-soluble UV-B filters are:

3-benzylidenecamphor derivatives, preferably 3-(4-methylbenzylidene)camphor, 4-aminobenzoic acid derivatives, preferably 2-ethylhexyl 4-(dimethylamino)benzoate, amyl 4-(dimethylamino)benzoate, polyethoxyethyl 4-bis(polyethoxy)aminobenzoate (available under the trade name Uvinul® P25 from BASF AG);
and UV-B filters bonded to polymers (e.g. benzylidene malonate polysiloxane, INCI: Polysilicone-15).

An example of a water-soluble UV-B filter advantageous according to the invention is 2-phenylbenzimidazole-5-sulfonic acid and its sodium, potassium or triethanolammonium salts.

For the purposes of the present invention, examples of UV-B filter substances liquid at room temperature are homomenthyl salicylate (homosalate), ethylhexyl salicylate, 2-ethylhexyl 2-cyano-3,3-diphenylacrylate (octocrylene), 2-ethylhexyl 2-hydroxybenzoate, esters of cinnamic acid, preferably 2-ethylhexyl 4-methoxycinnamate and isopentyl 4-methoxycinnamate and (3-(4-(2,2-bisethoxycarbonylvinyl)phenoxy)propenyl)methylsiloxane/dimethylsiloxane copolymer, which is available, for example, from DSM under the trade name Parsol® SLX.

Examples of UV broadband filters are derivatives of benzophenone, preferably 2-hydroxy-4-methoxybenzophenone (available under the trade name Uvinul® M40 from BASF AG), 2-hydroxy-4-methoxy-4′-methylbenzophenone, 2,2′-dihydroxy-4-methoxybenzophenone, 2-hydroxy-4-methoxybenzophenone-5-sulfonic acid (available under the trade name Uvinul® MS40 from BASF AG); triazines such as bisresorcinyltriazine derivatives, in particular 2,4-bis{[4-(2-ethylhexyloxy)-2-hydroxy]phenyl}-6-(4-methoxyphenyl)-1,3,5-triazine, which is available under the trade name Tinosorb® S from CIBA-Chemikalien GmbH, benzotriazoles, such as 2,2′-methylenebis(6-(2H-benzotriazol-2-yl)-4-(1,1,3,3-tetramethylbutyl)phenol), which is available under the trade name Tinosorb® M from CIBA-Chemikalien GmbH, 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane, which is produced by Chimex under the brand Mexoryl® XL, and [2,4′-dihydroxy-3-(2H-benzotriazol-2-yl)-5-(1,1,3,3-tetramethylbutyl)-2′-n-octoxy-5′-benzoyl]diphenylmethane, 2,2′-methylenebis[6-(2H-benzotriazol-2-yl)-4-(methyl)phenol], 2,2′-methylenebis[6-(2H-benzotriazol-2-yl)-4-(1,1,3,3-tetramethylbutyl)phenol], 2-(2′-hydroxy-5′-octylphenyl)benzotriazole, 2-(2′-hydroxy-3′,5′-di-t-amylphenyl)benzotriazole and 2-(2′-hydroxy-5′-methylphenyl)benzotriazole.

Further UV broadband filter substances are also inorganic pigments based on metal oxides and/or other metal compounds which are insoluble or sparingly soluble in water, in particular based on the oxides of titanium (TiO₂), zinc (ZnO), iron (e.g. Fe₂O₃), zirconium (ZrO₂), silicon (SiO₂), manganese (e.g. MnO), aluminum (Al₂O₃) or cerium (e.g. Ce₂O₃), mixed oxides of the corresponding metals, mixtures of such oxides, and barium sulfate. These pigments are X-ray amorphous or non-X-ray amorphous. They are particularly preferably pigments based on ZnO or TiO₂, in particular TiO₂.

The pigments can advantageously also be used in the form of commercially available oily or aqueous predispersions. Dispersion auxiliaries and/or solubilization promoters may advantageously be added to these predispersions.

The pigments used as broadband filters may advantageously be surface-treated (coated), the intention being, for example, to form and/or retain a hydrophilic, amphiphilic or hydrophobic character. This surface treatment can consist in providing the pigments with a thin hydrophilic and/or hydrophobic inorganic and/or organic layer by methods known per se. The various surface coatings may also comprise water. For the purposes of the present invention, inorganic surface coatings consist, for example, of aluminum oxide (Al₂O₃), aluminum hydroxide Al(OH)₃, or aluminum oxide hydrate, sodium hexametaphosphate (NaPO₃)₆, sodium metaphosphate (NaPO₃), silicon dioxide (SiO₂) or iron oxide (Fe₂O₃). These inorganic surface coatings may occur on their own, in combination and/or in combination with organic coating materials.

For the purposes of the present invention, organic coating materials consist, for example, of vegetable or animal aluminumstearate, vegetable or animal stearic acid, lauric acid, dimethylpolysiloxane (also: dimethicone), methylpolysiloxane (methicone), simethicones (a mixture of dimethylpolysiloxane with an average chain length of from 200 to 350 dimethylsiloxane units and silica gel), tri(m)ethoxycaprylylsilanes, diphenyl capryl methicone or alginic acid. These organic surface coatings may occur on their own, in combination and/or in combination with inorganic coating materials. Surface-modified zinc oxide particles and titanium dioxide particles suitable as UV broadband filters are commercially available, for example, under the name Z-COTE® HP1, Z-COTE® MAX or T-Lite™ SF, T-Lite™ SF-S or T-Lite™ Max from BASF AG.

Examples of approved commercial UV-B filters are 4-aminobenzoic acid, N,N,N-trimethyl-4-(2-oxoborn-3-ylidenemethyl)anilinium methylsulfate, homosalate, 2-phenylbenzimidazole-5-sulfonic acid and the sodium, potassium or triethanolammonium salts, alpha(2-oxoborn-3-ylidene)toluene-4-sulfonic acid and the sodium, potassium, or triethanolammonium salts, 2-ethylhexyl 2-cyano-3,3-diphenylacrylate (octocrylene), polyacrylamidomethyl benzylidene camphor, ethylhexyl methoxycinnamate, ethoxylated ethyl 4-aminobenzoate, isopentyl 4-methoxycinnamate, 2,4,6-trianilino-p-(carbo-2′-ethylhexyl-1′-oxy)-1,3,5-triazine (ethylhexyl triazone), diethylhexylbutamidotriazone, 3-(4′-methylbenzylidene)-DL-camphor, 3-benzylidenecamphor, 2-ethylhexyl salicylate, 2-ethylhexyl 4-dimethylaminobenzoate and dimethicodiethyl benzalmalonate.

Examples of approved commercial broadband filters, so-called UV-AB filters, are oxybenzone (benzophenone-3), drometrizole trisiloxane, 2-hydroxy-4-methoxybenzophenone-5-sulfonic acid and the sodium salt thereof (benzophenone-4), methylenebisbenzotriazolyl tetramethylbutylphenol, bisethylhexyloxyphenol methoxyphenyltriazine, zinc oxide and titanium dioxide.

In the method according to the invention, the UV-B filters and/or broadband filters, in particular UV-B filters, used are preferably those substances which have a high specific absorption in the UV-B region, preferably a specific absorption of A 1%/1 cm (absorption of a 1% strength solution at 1 cm path length) of at least 800, in particular of at least 1000.

Examples of particularly suitable highly absorbent UV-B filters are cinnamic acid derivatives, such as ethylhexyl methoxycinnamate or isoamyl p-methoxycinnamate, camphor derivatives, such as 3-(4′-methylbenzylidene)-DL-camphor, 2-phenylbenzimidazole-5-sulfonic acid and the sodium, potassium or triethanolammonium salts thereof, or triazine derivatives, such as ethylhexyltriazone or diethylhexylbutamidotriazone. Very particularly preferred UV-B filters are triazine derivatives, such as ethylhexyltriazone or diethylhexylbutamidotriazone.

The preparation produced by the method according to the invention preferably comprises, as UV-A filter, at least one highly absorbent substance such as, for example, hexyl 2-(4-diethylamino-2-hydroxybenzoyl)benzoate, BMDBM, terephthalidene dicamphor sulfonic acid (TDSA) and/or disodium phenyldibenzimidazoletetrasulfonate (DPDT), in particular hexyl 2-(4-diethylamino-2-hydroxybenzoyl)benzoate and/or BMDBM, and, as UV-B filter, at least one highly absorbent substance such as, for example, ethylhexyl methoxycinnamate, isoamyl p-methoxycinnamate, 3-(4′-methylbenzylidene)-DL-camphor, 2-phenylbenzimidazole-5-sulfonic acid and the sodium, potassium or triethanolammonium salts thereof, ethylhexyltriazone and/or diethylhexylbutamidotriazone, in particular ethylhexyltriazone or diethylhexylbutamidotriazone.

In addition to the UV-A and UV-B filter substances, these preparations specified above can also comprise broadband filters such as titanium dioxide and/or zinc oxide, preferably titanium dioxide, and/or also organic broadband filters, such as methylenebisbenzotriazolyltetramethylbutylphenol and/or bisethylhexyloxyphenolmethoxyphenyltriazine.

These combinations of UV-A and UV-B filters have very good efficiency and performance with regard to the ratio of SPF to the weight fraction of the UV filter substance used in the cosmetic and/or dermatological composition, i.e. preferably a ratio of SPF units to the percentage mass fraction of UV filters of at least 1.5, preferably of at least 2.

In the method according to the invention, very particular preference is given to using, as UV-A filter, an amino substituted hydroxybenzophenone, in particular hexyl 2-(4-diethylamino-2-hydroxybenzoyl)benzoate and, as UV-B filter, a triazine derivative, i.e. a symmetrically substituted triazine derivative, in particular 2,4,6-trianilino-p-(carbon-2′-ethylhexyl-1′-oxy)-1,3,5-triazine (Uvinul T150, BASF) or an asymmetrically substituted triazine derivative, such as diethylhexylbutamidotriazone, alone or mixtures of the two UV-B filters.

The ratio of the sum of the mass of the UV-A filters to the sum of the mass of the UV-B filters and broadband filters of at least 0.5 set in the method according to the invention can also be represented by the following formula:

Σ(m_{UV-A})/[Σ(m_{UV-B})+Σ(m_{UV-broadband})]≧0.5,

where

m_{UV-A} is the mass of individual UV-A filter,

m_{UV-B} is the mass of individual UV-B-filter, and

m_{UV-broadband} is the mass of the individual broadband filter.

Preferably, in the method according to the invention, the value for the ratio of the sum of the mass of the UV-A filters to the sum of the mass of the UV-B filters and broadband filters is in a range from 0.5 to 20, preferably in the range from 0.75 to 15, particularly preferably in the range from 0.9 to 10, especially in the range 1 to 6.

The sum of the mass of all UV-A filters in the formulation is usually up to 30% by weight, preferably up to 25% by weight. In particular, it is in a range from 0.5% by weight to 15% by weight.

The sum of the mass of all UV-B filters and broadband filters in the formulation is usually up to 40% by weight, preferably up to 25% by weight. In particular, it is in a range from 0.5% by weight to 20% by weight.

The cosmetic and/or dermatological preparations which can be prepared by the method according to the invention are generally constructed on the basis of a carrier which comprises at least one oil phase. However, preparations just on an aqueous basis when using compounds with hydrophilic substituents are also possible. Accordingly, oils, oil-in-water and water-in-oil emulsions, creams and pastes, lip and skin protection stick masses or grease-free gels, and sprays and foams are contemplated.

Suitable emulsions are, inter alia, also O/W macroemulsions, O/W microemulsions or O/W/O emulsions, where the emulsions are obtainable by phase inversion technology, as in DE-A-197 26 121.

Customary cosmetic auxiliaries which may be suitable as additives are, for example, coemulsifiers, fats and waxes, stabilizers, thickeners, biogenic active ingredients, film formers, fragrances, dyes, pearlizing agents, preservatives, pigments, electrolytes (e.g. magnesium sulfate) and pH regulators. Suitable coemulsifiers are preferably known W/O and in addition also O/W emulsifiers, such as, for example, polyglycerol esters, sorbitan esters or partially esterified glycerides. Typical examples of fats are glycerides; waxes to be mentioned are, inter alia, beeswax, paraffin wax or microwaxes, if appropriate in combination with hydrophilic waxes. Stabilizers which can be used are metal salts of fatty acids, such as, for example, magnesium stearate, aluminum stearate and/or zinc stearate. Suitable thickeners are, for example, crosslinked polyacrylic acids and derivatives thereof, polysaccharides, in particular xanthan gum, guar guar, agar agar, alginates and tyloses, carboxymethylcellulose and hydroxyethylcellulose, also fatty alcohols, monoglycerides and fatty acids, polyacrylates, polyvinyl alcohol and polyvinylpyrrolidone. Biogenic active ingredients are understood as meaning, for example, plant extracts, protein hydrolyzates and vitamin complexes. Customary film formers are, for example, hydrocolloids, such as chitosan, microcrystalline chitosan or quaternized chitosan, polyvinylpyrrolidone, vinylpyrrolidone-vinyl acetate copolymers, polymers of the acrylic acid series, quaternary cellulose derivatives and similar compounds. Suitable preservatives are, for example, formaldehyde solution, p-hydroxybenzoate or sorbic acid. Suitable pearlizing agents are, for example, glycol distearic esters, such as ethylene glycol distearate, but also fatty acids and fatty acid monoglycol esters. Dyes which can be used are the substances approved and suitable for cosmetic purposes, as are listed, for example, in the publication “Kosmetische Färbemittel” [cosmetic colorants] from the Dyes Commission of the German Research Society, published by Verlag Chemie, Weinheim, 1984. These dyes are usually used in concentrations of from 0.001 to 0.1% by weight, based on the total mixture.

An additional content of antioxidants is generally preferred. Thus, favorable antioxidants which may be used are all antioxidants which are customary or suitable for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof, (e.g. anserine), carotenoids, carotenes (e.g. β-carotene, lycopine) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl, and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximines, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrine), α-hydroxy acids (e.g. citric acid, lactic acid, malic acid), humic acid, bile acid, bile extracts, bilirubin, biliverdin, EDTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives thereof (e.g. ascorbyl palmitate, Mg ascorbyl phosphate, ascorbyl acetate), tocopherol and derivatives (e.g. vitamin E acetate, tocotrienol), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide).

Also advantageous are natural vegetable antioxidant complexes such as, for example, extracts of tea, grapes or algae, but also natural or nature-identical individual substances such as, for example, resveratrol.

Besides protecting the cosmetic and/or dermatological product against oxidation, antioxidants can also achieve antioxidative and also antiaging effects in the human skin.

For the purposes of the invention, very particular preference is therefore given to antioxidants which penetrate into the human skin and efficiently take effect there, and thus, in a certain sense synergistically to the photoprotective filters, protect the skin from damage by UV light, from sunburn and from reactive oxygen species and free radicals. Vitamin C and vitamin E and derivatives thereof are very particularly preferred.

The amount of the abovementioned antioxidants (one or more compounds) in the preparations is preferably 0.001 to 30% by weight, particularly preferably 0.05 to 20% by weight, in particular 1 to 10% by weight, based on the total weight of the preparation.

If vitamin E and/or derivatives thereof are used as antioxidant, it is advantageous to choose their respective concentration from the range from 0.001 to 10% by weight, based on the total weight of the formulation.

If vitamin A and/or derivatives thereof or carotenoids are the antioxidant or the antioxidants, it is advantageous to choose their respective concentration from the range from 0.001 to 10% by weight, based on the total weight of the formulation. Customary oil components in cosmetics are, for example, paraffin oil, glyceryl stearate, isopropyl myristate, diisopropyl adipate, cetylstearyl 2-ethylhexanoate, hydrogenated polyisobutene, Vaseline, caprylic/capric triglycerides, microcrystalline wax, lanolin and stearic acid. However, this list is illustrative and not exhaustive.

In addition, for the purposes of the present invention, natural and/or nature-identical and/or synthetic active substances with different active functions may be added to the preparations, such as, for example, caffeine for tightening the skin or promoting circulation, dihydroxyacetone and/or erythrulose for the purpose of self-tanning, bisabolol and/or panthenol for calming the skin and/or substances for moisture enrichment (moisturizing), for skin smoothing and in particular active substances for protecting against skin aging, such as, for example, vitamin A and/or derivatives thereof, plant extracts or else protein-like substances.

Further components of cosmetic and/or dermatological preparations for the purposes of the present inventions may fulfill additional functions, such as, for example, the coloring of the skin in decorative cosmetics, but also that of the product itself. Here, pigment-like, oil-soluble and/or water-soluble cosmetic color-imparting raw materials are generally used.

The total fraction of the auxiliaries and additives can be 1 to 80% by weight, preferably 6 to 40% by weight and the nonaqueous fraction (“active substance”) can be 20 to 80% by weight, preferably 30 to 70% by weight—based on the compositions. The compositions can be produced in a manner known per se, i.e. for example by hot, cold, hot-hot/cold or PIT emulsification. This is a purely mechanical process; there is no chemical reaction.

The compositions obtainable by the method according to the invention are thus in particular sunscreen preparations, which may be in liquid, paste or solid form, for example in the form of water-in-oil creams, oil-in-water creams or lotions, aerosol and pump foams, foam creams, gels, oils, grease sticks, powders, sprays or alcoholic-aqueous lotions.

Finally, further substances which absorb in the UV region and are known per se can be co-used provided they are stable within the entire system of the combination of UV filters to be used.

The present invention further provides the use of UV-A filters for increasing the sun protection factor in cosmetic and/or dermatological preparations which comprise at least one UV-B and/or broadband filter, wherein, in the finished cosmetic and/or dermatological preparation, the value for the ratio of the sum of the mass of the UV-A filters to the sum of the mass of the UV-B filters and broadband filters is at least 0.5, preferably in the range from 0.75 to 15, particularly preferably in the range from 0.9 to 10, especially in the range from 1 to 6.

Preferred UV-A filters for this use and preferred embodiments of the various other parameters and components have already been given in the preceding part of the description.

The invention further provides a photoprotective cosmetic and/or dermatological preparation comprising at least one UV-A filter and at least one UV-B filter, and if appropriate further cosmetic active ingredients, auxiliaries and additives, where the value for the ratio of the sum of the mass of the UV-A filters to the sum of the mass of the UV-B filters is at least 1, and the preparation comprises no broadband filters.

Preferred UV-A and UV-B filters have already been explained above in connection with the description of the method according to the invention for increasing the sun protection factor of a cosmetic and/or dermatological preparation.

It has likewise been explained above which compounds are defined as broadband filters.

Particular preference is given to a photoprotective cosmetic and/or dermatological preparation which comprises, as UV-A filter, a highly absorbent substance such as, for example hexyl 2-(4-diethylamino-2-hydroxybenzoyl)benzoate, BMDBM, terephthalidene dicamphor sulfonic acid (TDSA) or disodium phenyldibenzimidazoletetrasulfonate (DPDT), preferably hexyl 2-(4-diethylamino-2-hydroxybenzoyl)benzoate or BMDBM, in particular hexyl 2-(4-diethylamino-2-hydroxybenzoyl)benzoate, and which comprises, as UV-B filter, a highly absorbent substance such as, for example, ethylhexyl methoxycinnamate, isoamyl p-methoxycinnamate, 3-(4′-methylbenzylidene)-DL-camphor, 2-phenylbenzimidazole-5-sulfonic acid and/or sodium, potassium or triethanolammonium salts thereof, ethylhexyltriazone or diethylhexylbutamidotriazone, preferably ethylhexyltriazone or diethylhexylbutamidotriazone, in particular ethylhexyltriazone.

The value for the ratio of the sum of the mass of the UV-A filters to the sum of the mass of the UV-B filters in the preparation according to the invention is at least 1. Preferably, this value is between 1 and 10, in particular between 1 and 8.

The invention is explained by the following examples although these do not limit the invention.

### EXAMPLE 1

The components of phase A (see Table 1) were heated to 80° C. The components of phase B (see Table 1) were likewise heated to about 80° C. and stirred into phase A, at 80° C., with homogenization. The mixture of the two phases was cooled to about 40° C. with stirring. At about 40° C., phase C (Table 1) was added. The mixture was briefly homogenized again and then left to cool to room temperature with stirring.

TABLE 1

| Phase | Trade name | | % by wt. |
|---|---|---|---|
| A | Uvinul ® T 150 | Ethylhexyl Triazone | 2.00 |
| | Uvinul ® A Plus | Diethylamino Hydroxybenzoyl Hexyl Benzoate | 2.50 |
| | Cetiol B | Dibutyl Adipate | 8.00 |
| | Finsolv TN | C₁₂-C₁₅ Alkyl Benzoate | 8.00 |
| | Myritol 331 | Cocoglycerides | 12.00 |
| | Lanette E | Sodium Cetearyl Sulfate | 1.00 |
| | Eumulgin VL 75 | Lauryl Glucoside, Polyglyceryl-2 | 4.00 |
| | | Dipolyhydroxystearate, Glycerin | |
| | Lanette O | Cetearyl Alcohol | 2.00 |
| B | Edeta BD | Disodium EDTA | 0.05 |
| | Glycerin 87% | Glycerin | 3.00 |
| | Keltrol | Xanthan Gum | 0.30 |
| | Veegum Ultra | Magnesium Aluminum Silicate | 1.50 |
| | Water dem | Aqua dem | Ad 99.00 |
| C | Euxyl K 300 | Phenoxyethanol, Methylparaben, Butylparaben, | 1.00 |
| | | Ethylparaben, Propylparaben, Isobutylparaben | |

### EXAMPLES 2 to 20 AND COMPARATIVE EXAMPLES C1 AND C2

The following examples shown in Table 2 were prepared analogously as in Example 1.

TABLE 2

| Example | 1 | 2 | 3 | 4 | 5 | 6 | 7 | C1 | 8 | C2 | 9 |
|---|---|---|---|---|---|---|---|---|---|---|---|
| Uvinul ®→ | 2.5 | 5 | 10 | 2.5 | 7.5 | 2 | 10 | 2 | 10 | | |
| A Plus | | | | | | | | | | | |
| BMDBM | | | | | | | | | | 2 | 10 |
| Ethylhexyl | 2 | 2 | 2 | 2.5 | 2.5 | | | | | | |
| Triazone | | | | | | | | | | | |
| Diethylhexyl | | | | | | 2 | 2 | | | | |
| Butamido | | | | | | | | | | | |
| Triazone | | | | | | | | | | | |
| Octocrylene | | | | | | | | 5 | 5 | 5 | 5 |
| Tinosorb S | | | | | | | | | | | |
| TiO₂ | | | | | | | | | | | |
| ZnO | | | | | | | | | | | |
| Ratio Σ | 1.25 | 2.5 | 5 | 1 | 3 | 1 | 5 | 0.4 | 2 | 0.4 | 2 |
| UV-A/Σ | | | | | | | | | | | |
| (UV-B + UV- | | | | | | | | | | | |
| AB) | | | | | | | | | | | |
| SPF in vivo | 10 | 20 | 32 | 23 | 30 | 15 | 32 | 15 | 32 | 16 | 27 |
| Example | 10 | 11 | 12 | 13 | 14 | 15 | 16 | 17 | 18 | 19 | 20 |
| Uvinul ®→ | 3.5 | 10 | 3.5 | | 1.75 | 3.5 | 7 | 7 | 4 | 10 | 3.5 |
| A Plus | | | | | | | | | | | |
| BMDBM | | | | 3.5 | 1.75 | | | | | | |
| Ethylhexyl | | | 2.5 | 2.5 | 2.5 | 2 | 2 | 2 | 2.2 | 2.5 | 2 |
| Triazone | | | | | | | | | | | |
| Diethylhexyl | | | | | | | | | | | |
| Butamido | | | | | | | | | | | |
| Triazone | | | | | | | | | | | |
| Octocrylene | | | | | | | | | | | |
| Tinosorb S | 2 | 2 | | | | | | | | | |
| TiO₂ | | | | | | | | | 2 | | 1.5 |
| ZnO | | | | | | | | 3.5 | | 3 | |
| Ratio Σ | 1.75 | 5 | 1.4 | 1.4 | 1.4 | 1.75 | 3.5 | 1.27 | 0.95 | 1.82 | 1 |
| UV-A/Σ | | | | | | | | | | | |
| (UV-B + UV- | | | | | | | | | | | |
| AB) | | | | | | | | | | | |
| SPF in vivo | 13 | 19 | 20 | 22 | 23 | 14 | 19 | 28 | 30 | 43 | 19 |

The numerical data of the various UV filter substances are % by weight.

1-6. (canceled)

7. A method of increasing the sun protection factor of a cosmetic and/or dermatological preparation which comprises at least one UV-B and/or broadband filter comprising adding at least one UV-A filter during the production of said cosmetic and/or dermatological preparation, wherein the ratio of the sum of the mass of said at least one UV-A filter to the sum of the mass of said at least one UV-B filter and/or broadband filters in said cosmetic and/or dermatological preparation is at least 0.5.

8. The method of claim 7, wherein said at least one UV-A filter is a photostable or photostabilized organic UV-A filter.

9. The method of claim 7, wherein said at least one UV-A filter is an amino substituted hydroxybenzophenone and said at least one UV-B filter is a triazine derivative.

10. The method of claim 7, wherein the ratio of the sum of the mass of said at least one UV-A filter to the sum of the mass of said at least one UV-B filter and/or broadband filters in said cosmetic and/or dermatological preparation is in the range of from 0.5 to 20.

11. A photoprotective cosmetic and/or dermatological preparation comprising at least one UV-A filter and at least one UV-B filter and optionally further cosmetic active ingredients, auxiliaries, and additives, wherein the ratio of the sum of the mass of said at least one UV-A filter to the sum of the mass of said at least one UV-B filter in said photoprotective cosmetic and/or dermatological preparation is at least 0.5, and wherein said photoprotective cosmetic and/or dermatological preparation does not comprise a broadband filter.
